(19) United States
(12) Patent Application Publication
(10) Pub. No.: US 2022/0160576 A1
(43) Pub. Date: May 26, 2022

(54) PORTABLE HAIR AND SCALP TREATMENT DEVICE WITH CONDUCTIVE BRISTLES

(71) Applicant: L'OREAL, Paris (FR)

(72) Inventor: David B. Kosecoff, San Francisco, CA (US)

(73) Assignee: L'OREAL, Paris (FR)

(21) Appl. No.: 17/101,563

(22) Filed: Nov. 23, 2020

(51) Int. Cl. A61H 11/00; A61M 11/00; A61N 1/36; A61B 5/0531; A61B 5/0537; A61F 7/00; A61N 5/06; A46B 15/00; A46B 13/02; A46B 9/02

(52) U.S. Cl. CPC A61H 11/00; A46B 2200/104; A61N 1/36014; A61B 5/0531; A61B 5/0537; A61F 7/007; A61N 5/0625; A46B 15/0022; A46B 15/003; A46B 13/023; A46B 9/023; A46B 15/0034; A46B 15/0004; A61M 2205/0294; A61M 2205/0266; A61N 2005/0652; A61N 2005/0663; A61H 2201/1207; A61H 2201/0153; A61H 2201/105; A61M 11/005

(57) ABSTRACT

Brush portions are configured for use with a handheld device. The brush portion includes at least one bristle disposed on a bristle base, the bristle having an elongate first electrode, an elongate second electrode abutting the first electrode, and an insulator disposed between the first electrode and the second electrode. The first electrode and the second electrode are electrically connectable to a first voltage source and a second voltage source of the device, respectively.

### SUMMARY

Scalp and hair formulations exist for treating dandruff, hair-loss, stress reduction, itchiness, color and tint, oiliness, appearance, frizz, volume, shine, dryness, density, and more. However, smarter devices and methods for applying formulations to the scalp and hair are needed.

According to one representative aspect, the present disclosure provides a brush portion configured for use with a handheld device, and which can be fixed or removable from the device. The brush portion includes at least one bristle disposed on a bristle base. The bristle includes an elongate first electrode, an elongate second electrode abutting the first electrode, and an insulator disposed between the first electrode and the second electrode. The first electrode and the second electrode are electrically connectable to a first voltage source and a second voltage source of the device, respectively.

In any embodiment, the brush portion includes a first plurality of fluid conduits extending through the first electrode, the fluid conduits having a plurality of openings formed through at least one of an outer circumferential surface of the first electrode or an end tip surface of the first electrode.

In any embodiment, the brush portion includes a second plurality of fluid conduits extending through the second electrode and having a second plurality of openings formed through at least one of an outer circumferential surface of the second electrode or an end tip surface of the second electrode.

In any embodiment, the brush portion includes an electromagnetic energy source and/or a contact sensor disposed on at least one of the first electrode or the second electrode, and being configured to be electrically powered by the handheld device, e.g., via the first electrode and the second electrode.

In any embodiment, the brush portion includes at least one actuator disposed along the bristle in order to move the bristle when electrically powered by the handheld device. In any embodiment, the at least one actuator is at least partially formed of a piezoelectric material or a shape memory material. In any embodiment, the at least one actuator includes at least a first actuator and a second actuator disposed at diametrically opposed locations of the bristle.

In any embodiment, the first electrode is formed as a first half cylinder, and the second electrode is formed as a second half cylinder abutting the first half cylinder.

In any embodiment, the first electrode is formed as a first annular cylinder, and the second electrode is formed as a second cylinder disposed in an annular space of the first electrode.

In any embodiment, the first electrode is formed as a first annular half cylinder, the second electrode is formed as a second annular half cylinder abutting the first electrode, and the bristle further includes an inner cylinder disposed within an annular space formed by the first electrode and the second electrode.

According to another aspect, the present disclosure provides a device (e.g., a handheld personal cosmetic device) having a handle configured to receive a cartridge containing a formulation, and a brush portion according to any embodiment described herein.

In any embodiment, the handle includes a controller electrically connected to the brush portion, wherein the controller includes logic that when executed by the controller causes the device to perform operations, including: supplying the formulation from the cartridge to the bristle; and at least one of transmitting an electrical current between the first electrode and the second electrode; or measuring an electrical impedance between the first electrode and the second electrode.

In any embodiment, the device includes at least one actuator disposed along the bristle, and the controller includes logic that when executed by the controller causes the device to power the at least one actuator in order to move the bristle.

In any embodiment, the device includes an electromagnetic energy source and/or a contact sensor disposed on an end tip portion of the bristle, and the controller includes logic that when executed by the controller causes the device to power the electromagnetic energy source and/or contact sensor.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the present disclosure will become more readily appreciated as the same become better understood by reference to the following detailed description, when taken in conjunction with the accompanying drawings, wherein:

FIG. 1 is a partially exploded perspective view of a hair and scalp treatment device, according to a representative embodiment of the present disclosure.

FIG. 2 is a right elevation view of the hair and scalp treatment device of FIG. 1.

FIG. 3 is a rear elevation view of the hair and scalp treatment device of FIG. 1.

FIG. 4 is a perspective view of a bristle configured for use with the device of FIG. 1, in accordance with a representative embodiment of the present disclosure.

FIG. 5 is a perspective view of a bristle configured for use with the device of FIG. 1, in accordance with another representative embodiment of the present disclosure.

FIG. 6 is a perspective view of a bristle configured for use with the device of FIG. 1, in accordance with another representative embodiment of the present disclosure.

FIG. 7 is a perspective view of a bristle configured for use with the device of FIG. 1, in accordance with another representative embodiment of the present disclosure.

FIG. 8 is a perspective view of a bristle configured for use with the device of FIG. 1, in accordance with another representative embodiment of the present disclosure.

FIG. 9 is a schematic diagram of a device in accordance with a representative embodiment of the present disclosure, which is configured for use with any of the bristles of FIG. 4-FIG. 8.

### DETAILED DESCRIPTION

Individuals are washing hair with traditional wet water-based shampoo less and less frequently. A number of reasons can be offered for the reduction in this type of shampoo, such as preventing hair-loss and hair damage, and saving time and energy. Dry shampoo usage is on the rise, and consumers are prolonging intervals between salon visits to save money, leading to growing interest in home hair and scalp treatments.

In order to clean one's scalp without a shower or water, it is recommended that the user ‘preen’ their scalp with their fingers (to move oil and dirt from the scalp onto hair follicles) and follow-up with a moisturizer or serum to prevent dryness and/or hair loss. The application of such treatments involve imprecise dispensing, which can lead to excess drip. Pressing fingers into the scalp can transfer additional oils from the fingers onto the scalp and/or make the fingers feel oilier. Furthermore, dry shampoo is not useful for cleaning the scalp, as it often contains alcohol that can dry the scalp.

Scalp treatment and scalp-directed formulations are commonly applied via pipettes, foams or powders, and require a user to manually part the hair. Powders and foams are messy because they get on hands. Further, dripping excess product onto scalp can create runoff and greasy-looking hair.

Accordingly, there is a need for an improved device that precisely applies formulation to a user's scalp and/or hair.

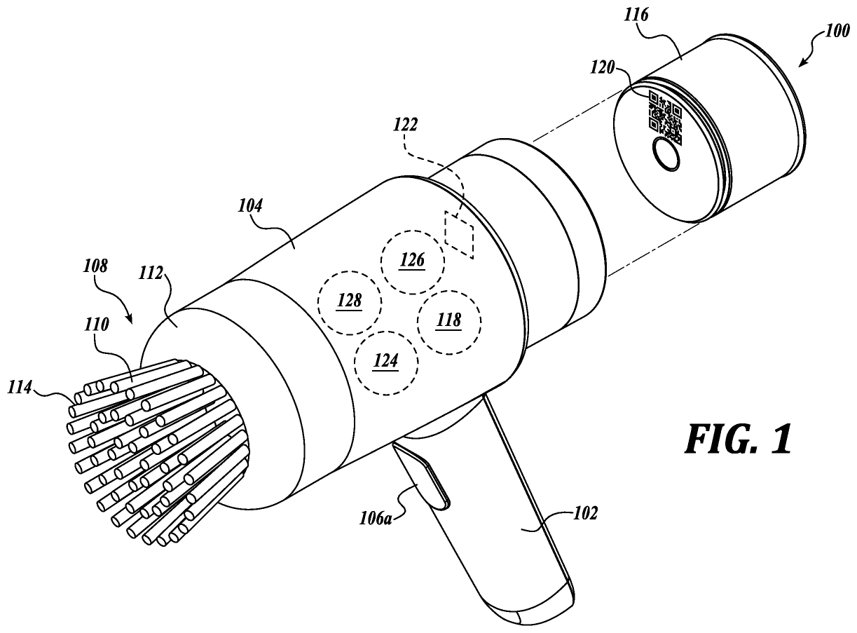

FIG. 1-FIG. 3 show a handheld device 100 (hereinafter, a “device”) configured for applying a formulation (e.g., a scalp treatment or a hair treatment), and which has additional functionality through the individual activation of bristles and/or teeth for dispensing, sensing, massaging, and/or other uses. In an embodiment, the device 100 has brush- or comb-like architecture configured to provide any number of functionalities, including supplying formulation to a user's scalp and/or hair, stimulating the user's scalp, verifying contact between the bristles/teeth and the scalp, measuring a moisture content of the scalp, applying heat and/or light therapy to the scalp, curing formulation applied the scalp and/or the hair, massaging the scalp, and/or removing excess formulation and/or unwanted particulates. The device 100 enables intuitive action, which provides a familiar gesture that is easy for a user to incorporate into her/his current beauty and haircare routines.

Device 100 includes a handle 102 comprising a cylindrical body 104. One or more buttons 106a, b located on handle 102 toggle any of the features described herein. The present disclosure is not limited to devices 100 having a shape as shown in FIG. 1-FIG. 3.

Brush portion 108 is fixed to the body 104 in the illustrated embodiment; however in some embodiments, brush portion 108 is removable from the body 104, e.g., as a modular piece configured to be interchanged with other compatible brush portions and/or comb portions having different features and functionalities, which are described below. Accordingly, the present disclosure provides a device 100 comprising a plurality of different modular brush portions 108, each having a different configuration in accordance with any of the embodiments of the present disclosure.

Brush portion 108 includes a plurality of bristles or teeth, e.g., bristle 110 disposed on a bristle base 112. Each bristle terminates in a tip 114. Device 100 is shown with a brush configuration in the FIGURES; however, this is not limiting. For example, device 100 is configured with a comb configuration in some embodiments, e.g., a single row of teeth. Indeed, as used herein, the term “brush portion” includes both brushes and combs. Likewise, the term “bristle” includes both “bristles” (of a brush) and “teeth” (of a comb).

As described in detail below, in some embodiments, one or more of the bristles of the brush portion 108 has a fluid conduit therethrough configured to deliver formulation to a user's scalp, and also includes one or more of the following features:
electrically conductive bristles stimulate a user's scalp with safe electrical current;
electrically conductive bristles aid in the detection of contact between the bristles and the user's scalp by functioning as sensing electrodes, in connection with impedance measuring circuitry;
electrically conductive bristles aid in the determination of a moisture level of the user's scalp, in connection with impedance measuring circuitry;
electrically conductive bristles provide power (from the device) to one or more electromagnetic energy sources that treat a user's scalp, hair, or formulation applied thereon with thermal and/or electromagnetic energy;
bristles massage a user's scalp; and/or
bristles aid in vacuuming away debris and excess formulation away from the user's scalp.

Device 100 is configured to removably receive a formulation cartridge 116 in a receiving portion thereof, e.g., a recess disposed in a rear end of the body 104. The cartridge 116 contains a formulation, such as a scalp treatment or hair treatment. Said formulation is provided to one or more of the tips via one or more fluid conduits extending through the body 104. In one embodiment, the formulation is provided through the body 104 to the tips via a formulation dispenser 118, described below.

Device 100 enables cartridges 116 to be easily swapped by a user, in order to provide different formulations or to replace depleted formulation. In some embodiments, cartridge 116 is a disposable consumable element which is discarded or recycled after the formulation stored therein is depleted. In other embodiments, the cartridge 116 is re-Tillable. In some embodiments, device 100 is configured to hold a plurality of cartridges 116, wherein each cartridge 116 is filled with a different formulation. Alternatively, the present disclosure provides methods that include the application of a first formulation from a first cartridge 116 and then a second, different, formulation from a second cartridge 116 as part of a scalp or hair treatment routine.

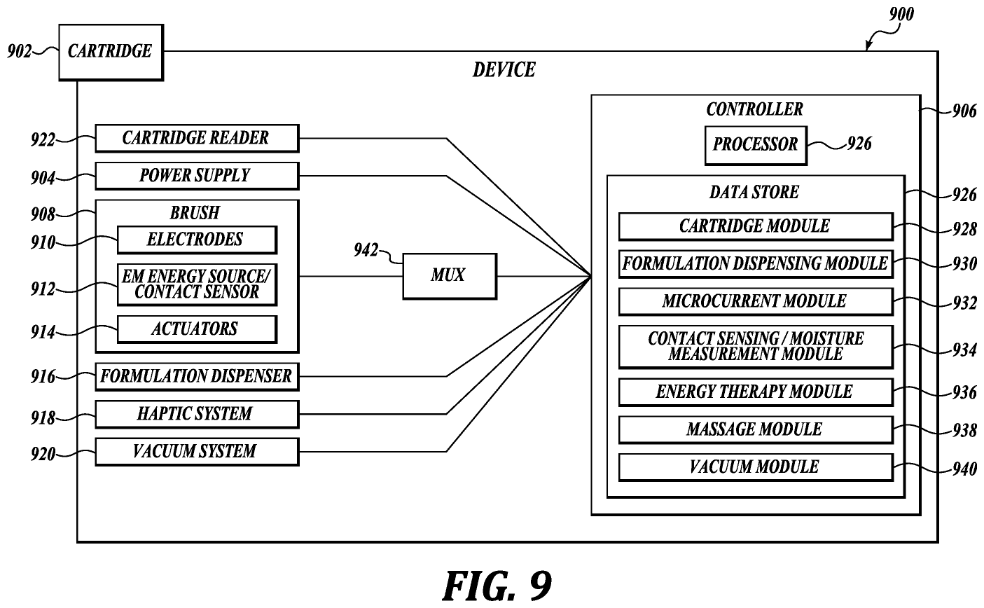

In some embodiments, cartridge 116 has a product identification tag 120 encoded with instructions for one or more operating parameters of the device 100 based on the specific formulation contained in the cartridge 116. Accordingly, some embodiments of device 100 include a product identification tag reader 122 configured to read the product identification tag 120 and to process the encoded signals into instructions for operation and control of the device 100 based on the particular formulation (e.g., in connection with a controller as described below with respect to FIG. 9).

Representative product identification tags 120 include bar codes, QR codes, RFID codes, and the like. Product identification tag 120 is encoded with machine readable signals that convey the device settings for the particular formulation, for example any one or more of the following: formulation dispensation time; formulation droplet size (fine, coarse); massage; electrical current application; vacuum; pattern formation (e.g., flat fan versus cone, wide versus narrow, solid versus hollow, stream versus mist); energy source activation time; and the like.

Formulation dispenser 118 is disposed in the body 104 and supplies the formulation from the cartridge 116 to the bristles 110 of the brush portion 108, in particular to the tips of the bristles. Accordingly, the formulation dispenser 118 includes one or more fluid conduits, manifolds, and/or pumps fluidly connecting the cartridge 116 to the bristles.

Formulation dispenser 118 is configured to dispense one or more formulations through the tips of the bristles as a fine mist, a liquid, and/or any form in-between. In an embodiment, formulation dispenser 118 includes a compressor, pump, and/or a nebulizer to generate a mist from the formulation. In the case of a pump or compressor, formulation dispenser 118 causes air or the formulation to flow at a high velocity, which propels the formulation through fluid conduits in the bristles 110 as described below. In some embodiments, a single, centralized formulation dispenser 118 is disposed in the body 104 of device 100.

In some embodiments, formulation dispenser 118 includes a nebulizer that generates a mist or vapor from the formulation and dispenses the same through individual bristles. This has the advantages of gentle dispersion of the formulation, reduction of waste, and improved coverage control. In some embodiments, the nebulizer is an ultrasonic wave generator that produces a mist from the formulation. Representative ultrasonic wave generators include, for example, vibrating diaphragm-type nebulizers that generate vibration in one or more ultrasound frequencies (e.g., between over 20 KHz, such as 1 MHz or greater). In some embodiments, the nebulizer utilizes a piezoelectric material.

Some embodiments of device 100 include a haptic system 124. Some such haptic systems include a massage therapy system configured to vibrate one or more of the bristles in order to stimulate the scalp. In some embodiments, the haptic system 124 includes the bristles 110 or elements thereof, such as one or more piezoelectric or shape memory actuators disposed in or along the bristles, which actuators move the bristles when electrically powered by the handle 102.

In some embodiments, device 100 is configured to provide one or more of a heating therapy and/or light therapy through the bristles, in order to generate heat that treats the scalp and/or hair either alone or together with the dispensing of formulations from the cartridge 116. Accordingly, some embodiments of device 100 include one or more electromagnetic energy sources (e.g., an LED, laser diode, incandescent device, halogen device, or similar energy source) disposed on one or more of the bristles (in particular the tips thereof), in order to deliver thermal energy and/or light energy to the scalp.

Some embodiments of device 100 include a vacuum system 126 having a vacuum generating motor and collector. In one embodiment, the vacuum motor is a variable speed motor connected to impeller vanes that cause a stream of air to enter one or more of the bristles 110 via fluid conduits formed therein. The vacuum motor induces a stream of air to enter through said conduits, in order to carry away the used formulation along with any debris and oils from the scalp and hair, which then collect in the collector. In some embodiments, the collector includes a vent disposed on the body 104 of the device 100. The vent allows the stream of air to exit the device 100, while the used and debris become trapped in the collector.

The foregoing features and systems are operatively (i.e., electrically) connected or connectable to an on-board controller 128, which is described in further detail below.

Additional features of the device 100 will become apparent in view of the details provided below.

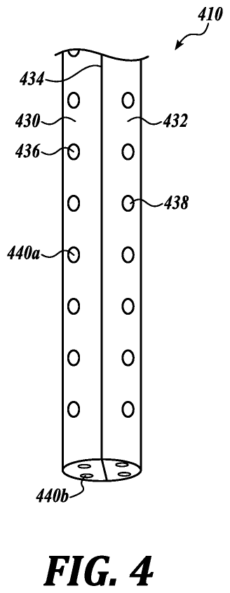

FIG. 4-FIG. 8 show different embodiments of electrically conductive bristles of the present disclosure. All of the following conductive bristles are configured to be provided on the brush portion of any of the devices described herein (e.g., disposed on a bristle base 112 of the device 100 of FIG. 1). Moreover, the present disclosure provides brush portions having a plurality of the bristle structures described herein.

FIG. 4 shows one representative conductive bristle 410 having an elongate first electrode 430 formed as a first hollow half cylinder, and a second electrode 432 formed as a hollow half cylinder abutting the first electrode. The first electrode 430 and second electrode 432 are each at least partially made from an electrically conductive material (e.g., a metal such as copper), or include an electrically conductive pathway therethrough which is formed of a conductive material. Accordingly, the first electrode 430 and second electrode 432 are configured to electrically connect to a first and second voltage source of the device, e.g., pins of the on-board controller, a multiplexer, and/or power supply. When so connected to the device, one of the first electrode 430 and second electrode 432 is a positive electrode, while the other is a negative electrode.

A thin electrical insulator 434 (e.g., 0.1 mm-2.0 mm thick) extends along the length of the bristle 410 and separates first electrode 430 from second electrode 432 such that no part of first electrode 430 electrically contacts the second electrode 432. Insulator 434 is formed of a polymer, a ceramic, or similar insulator. In some embodiments, insulator 434 is formed of a dielectric material. To clarify, first electrode 430 abuts second electrode 432 despite the presence of the insulator 434 therebetween. In some embodiments, a plurality of such insulators 434 are utilized to separate first electrode 430 from second electrode 432. Whereas the illustrated bristle 410 has a cylindrical shape, other embodiments have other elongate shapes, including oblong, rectangular, square, and other polygonal shapes.

Thus, in some embodiments, when a voltage differential is applied across the first electrode 430 and second electrode 432 and when a conductive pathway (e.g., a user's scalp, acting as a ground path) electrically connects the two electrodes, electrical current (e.g., on the order of microamperes) travels from the first electrode 430 to the second electrode 432. In other embodiments, first electrode 430 and second electrode 432 are configured to be used as electrodes for measuring impedance across a user's scalp (e.g., to measure a scalp moisture level and/or to verify contact between the bristle 410 and the user's scalp), in connection with complementary impedance measurement features described herein. In still other embodiments, the first electrode 430 and second electrode 432 are terminals for one or more electrical loads disposed on the bristle 410 and powered by the power supply, e.g., an electromagnetic energy source such as an LED. To clarify, the electrodes of any of the bristles described herein are configured to operate as electrodes or terminals for one or more of the previously-described functionalities in connection with multiplexing and/or firmware provided in the device and described below.

In the illustrated embodiment, the bristle 410 is not provided with a conductive pathway connecting the first electrode 430 and second electrode 432. Rather, when the bristle 410 is placed against a user's scalp, the scalp itself provides the conductive pathway between the two electrodes. Thus, when the first electrode 430 and second electrode 432 are electrically connected to first and second voltage sources of the device, electrical current is transmitted through the user's scalp between the first electrode 430 and second electrode 432, thus providing a stimulating effect.

The bristle 410 is configured to facilitate application of one or more formulations to the user's hair and/or scalp. Such formulation is provided to the bristle 410 from the cartridge of the device and routed through the formulation dispenser of the device. To carry the formulation to the user's scalp and or near the roots of the user's hair, bristle 410 has optional fluid conduits formed therein, including internal fluid conduits and/or openings formed on through an exterior surface. For example, first electrode 430 (i.e., the first hollow half cylinder) has first fluid conduits 436 formed therethrough, including openings formed along an exterior surface thereof. Likewise, second electrode 432 has second fluid conduits 438 formed therethrough, including openings formed along the exterior surface thereof.

Openings 440a, b are respectively formed through an outer circumferential surface (to apply formulation to a user's hair) and through an end tip surface of the first electrode 430 (to apply formulation to a user's scalp and/or roots). Second electrode 432 likewise has similar openings. Such outer circumferential openings are distributed evenly across the outer circumferential surface in some embodiments, and in other embodiments have a greater opening density in a localized portion of the outer circumference, e.g., near the end tip portion to concentrate formulation near a user's roots.

In some embodiments:
bristle 410 is provided with openings only along the outer circumferential surface or through an end tip surface, but not both;
bristle 410 is not provided without any fluid conduits or openings;
the fluid conduits and openings are laser-cut openings or cast openings;
the fluid conduits of the first electrode 430 are fluidically separate from the fluid conduits of the second electrode 432. In this way, two different formulations can be delivered to the user's scalp and/or hair.

In some embodiments, first and/or second electrodes 430, 432, and/or first and/or second fluid conduits 436, 438 are made from or embedded with a shape memory or piezoelectric material configured for actuation by an electric current provided by the device, for the benefit of controlling a direction of movement of the bristle 410. Such materials include polymer-, ceramic-, and alloy-shape memory materials, and the like.

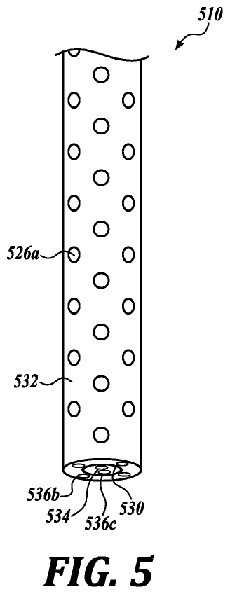

FIG. 5 illustrates another representative bristle 510, which includes the features of bristle 410 except where described below.

Bristle 510 has cylinder-in-cylinder construction. That is, an inner cylinder is disposed within an annular space formed by a larger-diameter outer cylinder, e.g., in a coaxial configuration. Here, although the bristle 510 is in the shape of a “cylinder,” other similar embodiments have different cross-sectional shapes, including triangular, rectangular, square, or any other polygonal cross sectional shape.

Here, the first electrode 530 is formed as the outer cylinder, and the second electrode 532 is formed as the inner cylinder, and the two are separated by a cylindrical insulator 534.

Bristle 510 is provided with fluid conduits, similar to the bristle 410 of FIG. 4. In particular, first electrode 530 is provided with fluid conduits disposed through an outer circumferential surface thereof (e.g., fluid conduit 536a) and through an end tip surface thereof (e.g., fluid conduit 536b). By comparison, first electrode 530 is provided only with openings through an end surface thereof. In some embodiments, such end tip openings are provided through a perforated flat or domed disk having small openings therethrough (e.g., fluid conduit 536c). In some embodiments, the fluid conduits of first electrode 530 are fluidically separate from the fluid conduits of second electrode 532.

In some embodiments, first and/or second electrodes 530/532 and/or the fluid conduits thereof are made from or embedded with a shape memory or piezoelectric material configured for actuation by an electric current provided by the device, for the benefit of controlling a direction of movement of the bristle 510. In some embodiment, the shape memory and piezoelectric materials are fabricated as coils, which are effective for actuating the bristle 510 vertically along the Z axis (i.e., in the axial direction of the bristle and coil).

Thus, the bristles of FIG. 4 and FIG. 5 are both configured to apply electrical current to a user's scalp when connected to first and second voltage energy sources of the device. Additionally, the bristles are configured to be used as electrodes for measuring impedance across a user's scalp, in connection with impedance measurement features described herein.

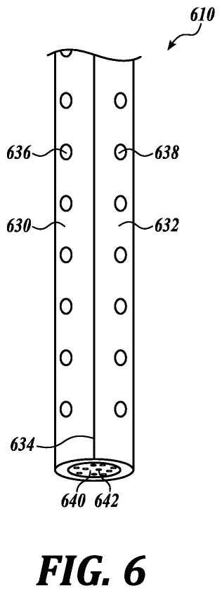

FIG. 6 illustrates a bristle 610, which includes the features of bristles of FIG. 4 and FIG. 5 except where described below.

Bristle 610 is constructed similarly to bristle 410 of FIG. 4 in that it has a first electrode 630 formed as a first hollow (annular) half cylinder, mated with a second electrode 632 formed as a hollow (annular) half cylinder. The second first electrode 630 and second electrode 632 are separated by an electrical insulator 634. Similar to the bristle 510 of FIG. 5, bristle 610 has an inner cylinder 640 (not an electrode) which fits within the annular space formed by the first electrode 630 and second electrode 632.

First electrode 630 has first fluid conduits 636 formed therethrough and through a circumferential outer surface thereof. Likewise, second electrode 632 has second fluid conduits 638 formed therethrough and through a circumferential outer surface thereof. Inner cylinder 640 has fluid conduits 642 formed on an end tip surface thereof, i.e., to direct formulation to a user's scalp and/or roots.

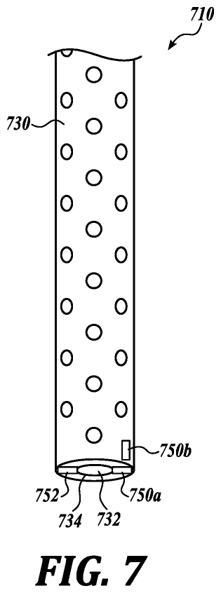

FIG. 7 illustrates a bristle 710, which includes the features of bristle 510 of FIG. 5 except where described below. In particular, bristle 710 has an annular first electrode 730 formed as an outer cylinder, a second electrode 732 formed as an inner cylinder that fits within the annular space formed by the first electrode 730, and an insulator 734 interposed between the two electrodes.

A plurality of optional energy sources 750a-b are disposed on the bristle 710. In particular, energy source 750a is disposed on an end tip portion of the bristle 710, and is electrically connected to both the first electrode 730 and second electrode 732, which function as positive and negative terminals of the energy sources, or vice versa. Accordingly, the energy sources are configured to be powered by the device via the first electrode 730 and second electrode 732. In some embodiments however, one or more energy sources are not powered via the first electrode and the second electrode. Generally speaking, the energy sources are electrical loads configured to deliver thermal energy and or electromagnetic energy (e.g., ultraviolet or visible light energy) to a user's scalp, hair, and/or formulation provided thereon. This provides a number of benefits to the user, including soothing heat therapy, stimulating hair follicles, improved formulation penetration, curing of formulation applied to the scalp and/or hair, and other benefits.

Accordingly, each energy source is a light emitting diode (LED), laser diode, incandescent device, halogen device, or similar energy source. In some embodiments, a plurality of energy sources are provided on the bristle 710, for example at the end tip portion, and/or disposed around the outer circumferential surface of the first electrode 730 (in particular at a distal end thereof near the end tip portion, as in the case of energy source 750b). In some embodiments, the energy source 750a is configured to be driven on the order of volts or microvolts, for safety and in accordance with regulations.

Advantageously, placing one or more energy sources at or near the end tip portion of bristle 710 and utilizing the first electrode 730 and second electrode 732 to drive the same provides better performance (in particular greater energy intensity at higher power efficiency) than positioning the energy source at the base of the bristle or by delivering light energy through a fiber optic pathway from a driver disposed in the body of the device, or by delivering thermal energy along a conductive pathway disposed along the bristle.

The number, size, and position of the energy sources 750a-b is representative, not limiting. Some embodiments includes a single energy source (e.g., disposed on an end tip portion of bristle 710). Some embodiments include two, four, or more energy sources.

In some embodiments, one or more of the energy sources is an LED. Such LEDs can be configured to provide light at a single wavelength and/or frequency (e.g., a laser LED) or at a range of wavelengths and/or frequencies. In some embodiments, one or more of the energy sources is configured to provide light over a broad range of the electromagnetic spectrum (frequency and/or wavelength). In some embodiment, one or more of the energy sources includes one or more Group III-V (GaAs) based LEDs configured to emit electromagnetic radiation at wavelengths in a range spanning from green visible light to near infrared.

In some embodiments, one or more of the energy sources includes one or more Group III-nitride blue LED solid state emitters configured to emit electromagnetic radiation at wavelengths in a range spanning from ultraviolet to blue visible light. The energy emitted from the energy source in the foregoing embodiments has been shown to treat scalp conditions, to stimulate the cells of hair follicles, and to provide other beneficial purposes. For further benefit, in some embodiments, the intensity of the energy emitted by one or more of the energy sources is configured to be varied by controlling the current from the device.

In some embodiments, the wavelength output of one or more of the energy sources includes one or more gallium-indium-nitrogen (GaInN) LEDs that have a wavelength output of about 360-370 nm. In other embodiments, one or more of the energy sources emit electromagnetic energy in a range of wavelengths from about 200 nm to about 2000 nm, which includes wavelengths in the ultraviolet range (about 350 nm) and near infrared (about 1200 nm).

Bristle 710 also includes an optional contact sensor 752 disposed on the end tip portion between the first and second electrodes 730, 732 for the advantage of verifying scalp contact. In some embodiments, contact sensor 752 is a dielectric material 752 that enables the bristle 710 to function as a dielectric contact sensor (a capacitance detector). In some embodiments, contact sensor 752 is a piezoelectric material electrically connected to first and second electrodes 730, 732. The shape and size of contact sensor 752 is representative, not limiting. For example, in some embodiments, the contact sensor 752 is a flat disk. Additionally, some embodiments include more than one contact sensor disposed on an end tip portion. In some embodiments, contact sensor 752 is a dielectric material formed integrally with insulator 734 of a same material.

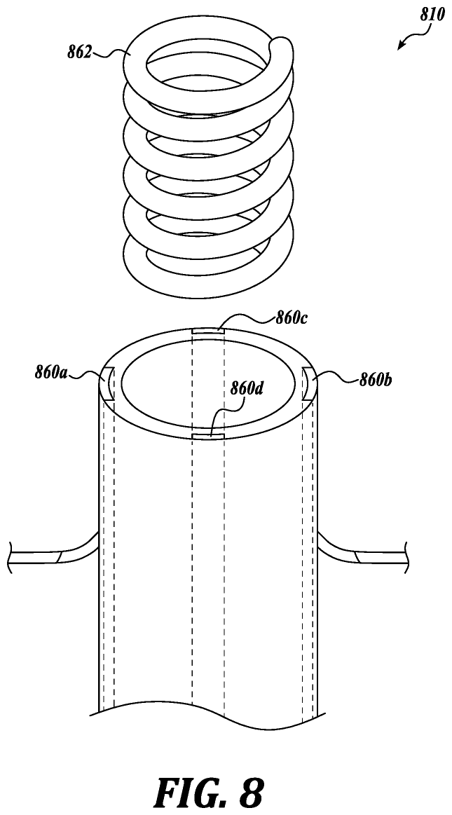

FIG. 8 illustrates a bristle 810, which has features that are adaptable to any of the foregoing bristles, and which is particularly adapted for use with a haptic system of a device.

A first pair of piezoelectric or shape memory actuators 860a, 806, are disposed (placed or embedded) along the length of the cylinder of the bristle 810 in diametrically opposed locations (e.g., along an outer surface of the bristle 810). The actuators 860a, b are configured to be actuated one at a time to cause a side-to-side oscillatory motion relative to a normative center portion of the bristle 810, e.g., along an x-axis. The bristle 810 includes an optional second pair of actuators 860c, d disposed at diametrically opposed locations, and separated by ninety degrees from actuator 860a, b, such that they can be actuated one at a time to create a side-to-side motion oscillatory motion, such as along a y-axis.

In some embodiments, the actuators are each constructed from a piezoelectric material in order to produce contractions when powered by the device (e.g., when a voltage is applied). In this manner, side-to-side actuation is possible in both the x- and/or y-axes.

In some embodiments, the bristle 810 is configured to oscillate along the Z-axis, i.e., along the length of the bristle 810. In some such embodiments, the top end of the bristle 810 (i.e., the end nearest the device) rests against a piezoelectric or shape memory coil 862 configured to be actuated by a voltage provided by the device to oscillate the bristle 810 up and down.

The number and placement of actuators in FIG. 8 is representative, not limiting. Additional configurations are possible, for example bristles having one, two, three, or five or more such actuators. Although representative embodiments using piezoelectric and shape memory materials are illustrated, the present disclosure includes other configurations. For example, in some embodiments, piezoelectric materials are tubes or stacked in a configuration that causes up and down vibration. As another example, in some embodiments, shape memory materials are provided as strips configured to cause side-to-side, bending, and/or shearing motions for X- and Y-axis vibration. In any of the bristles described herein, any combination of one or more piezoelectric or shape memory materials can be used to provide the tips with vibration in one or more axes.

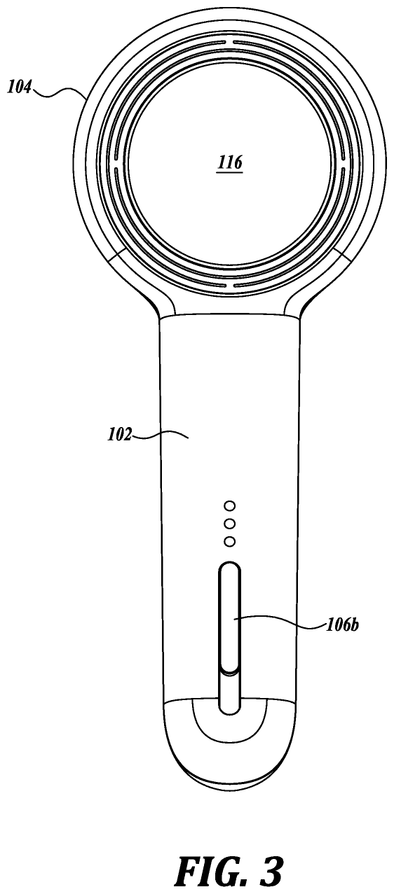

Turning now to FIG. 9, a device 900 is shown schematically in order to understand the main systems. Device 900 is representative of the device 100 of FIG. 1, and is compatible with any of the bristle structures described herein in FIG. 3-FIG. 8. Furthermore, device 900 is configured to receive one or more cartridges 902. In some embodiments, device 900 includes the cartridge 902.

Device 900 is powered by a power supply 904, which in some embodiments is an alternating current (AC) power supply, such as common household alternating current that utilizes an electrical cord (not shown) to supply power to the device 900. In other embodiments, power supply 904 is a direct current (DC) power supply, such as a rechargeable battery configured to be charged by plugging into a household alternating current outlet. Device 900 is electrically connected directly or indirectly to any one or more of the systems requiring power, namely a controller 906, which itself is electrically connected to numerous other systems of device 900.

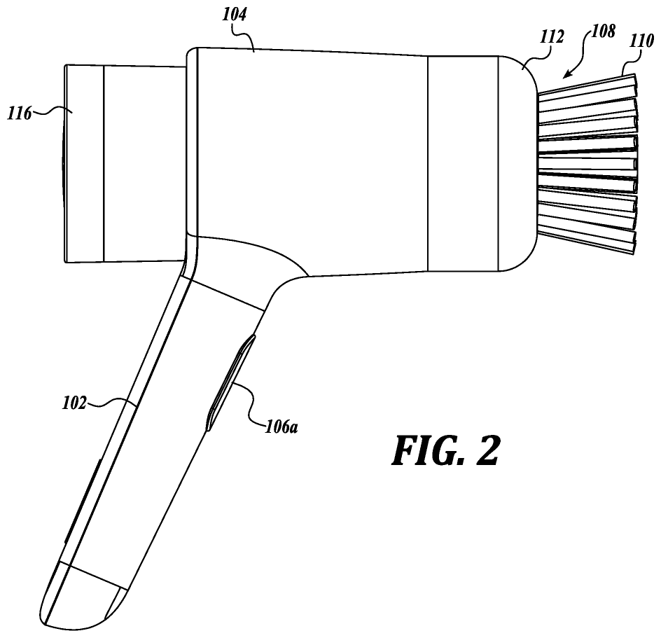

Brush portion 908 includes one or more bristles as described in FIG. 2-FIG. 8. Accordingly, each bristle includes one or more of: a plurality of electrodes 910; an electromagnetic energy source and/or contact sensor 912; and/or an actuator 914. In some embodiments, brush portion 908 is a permanent fixture attached to a housing of device 900; however, in other embodiments, brush portion 908 is a module fixture reversibly attachable to the housing.

Formulation dispenser 916 is as described above with respect to FIG. 1. For example, in some embodiments, formulation dispenser 916 is a nebulizer that provides a formulation from cartridge 902 to the brush portion 908, and in particular one or more bristles thereof.

Haptic system 918 is as described above with respect to FIG. 1. For example, in some embodiments, haptic system 918 includes one more piezoelectric or shape memory actuators 914 configured to move one or more of the bristles relative to a normative center, e.g., oscillatory movement along an x-axis, y-axis, and/or z-axis. In some embodiments, haptic system 918 includes one or more additional motors, piezoelectric actuators, drivers, or other devices configured to cause motion in the device 900.

Vacuum system 920 is as described above with respect to FIG. 1. For example, in some embodiments, vacuum system 920 includes a vacuum motor configured to draw ambient air through fluid conduits in one or more bristles, in order to remove debris from a user's scalp. Said debris is collected in a collector (e.g., a removable bin), which a user can periodically empty.

A cartridge reader 922, e.g., an RFID reader or nearfield reader, is disposed in device 900 proximate to where the device 900 receives cartridge 902 and positioned to read information contained in a product identification tag of the cartridge 902, as described above.

To clarify, in some embodiments, parts of the bristles also form part of the formulation dispenser 916, haptic system 918, and/or vacuum system 920.

A controller 906 is operatively connected (e.g., electrically connected) to the power supply 904, the brush portion 908 (in particular, to the electrodes and actuators thereof), the formulation dispenser 916, haptic system 918, vacuum system 920, and cartridge reader 922.

Controller 906 includes a processor 924 (e.g., a general processing unit, graphical processing unit, or application specific integrated circuit); a data store 926 (a tangible machine-readable storage medium); and a plurality of modules that may be implemented as software logic (e.g., executable software code), firmware logic, hardware logic, or various combinations thereof. In some embodiments, controller 906 includes a communications interface having circuits configured to enable communication with other systems of the device 900, including the brush portion 908, formulation dispenser 916, haptic system 918, vacuum system 920, cartridge reader 922, a remote server, a base station, and/or other network element via the internet, cellular network, RF network, Personal Area Network (PAN), Local Area Network, Wide Area Network, or other network. Accordingly, the communications interface may be configured to communicate using wireless protocols (e.g., WIFI®, WIMAX®, BLUETOOTH®, ZIGBEE®, Cellular, Infrared, Nearfield, etc.) and/or wired protocols (Universal Serial Bus or other serial communications such as RS-234, RJ-45, etc., parallel communications bus, etc.). In some embodiments, the communications interface includes circuitry configured to initiate a discovery protocol that allows controller 906 and other network element (e.g., the cartridge 902 and/or brush portion 908) to identify each other and exchange control information (e.g., dispensation time for the formulation stored in the cartridge 902 or other operating parameter). In an embodiment, the communications interface has circuitry configured to a discovery protocol and to negotiate one or more pre-shared keys.

Data store 926 is a tangible machine-readable storage medium that includes any mechanism that provides (i.e., stores) information in a non-transitory form accessible by a machine (e.g., the device 900, a smartphone, computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

Controller 906 includes a plurality of modules. Each module includes logic that, when executed by processor 924, causes the device 900 to perform one or more operations related to treatment of the scalp and/or hair of a user.

Cartridge module 928 identifies information stored within the product identification tag of the cartridge 902, for example, the formulation identification, formulation expiration date, and/or device settings corresponding to the particular formulation, for example any one or more of the following: formulation dispensation time; formulation droplet size (fine, coarse); massage; electrical current application; vacuum; pattern formation (e.g., flat fan versus cone, wide versus narrow, solid versus hollow, stream versus mist); energy source activation time; and the like. Said information is communicated to one or more other modules (namely the formulation module 930) in order to properly adjust the device settings.

Formulation module 930 operates the formulation dispenser 916, e.g., in a manner consistent with the information stored within the product identification tag of the cartridge 902. For example, the formulation module 930 operates one or more nebulizers, ultrasonic wave generators, pumps, and/or drivers of the formulation dispenser 916 in order to provide the formulation from the cartridge 902 to one or more bristles of the brush portion 908. In some embodiments, the formulation module 930 modulates one or more of the following operational parameters: formulation dispensation time; formulation dispensation pressure; formulation droplet size (fine, coarse); pattern formation (e.g., flat fan versus cone, wide versus narrow, solid versus hollow, stream versus mist); and the like. In some embodiments, formulation module 930 receives a signal from the contact sensing/moisture measurement module 934 (described below) indicative of the impedance of the user's scalp, which indicates contact between the bristles and scalp. Accordingly, the formulation module 930 starts/stops, or increases/decreases a flow of the formulation based upon the received impedance value.

Microcurrent module 932 powers the electrodes 910 of one or more bristles of the brush portion 908, in order to apply electrical current to a user's scalp. In some embodiments, microcurrent module 932 modulates one or more of the following operational parameters: current value, current duration, and/or the identity of the specific bristles applying/receiving the electrical current. As to the latter feature, in some embodiments, the microcurrent module 932 powers different bristles at different times, in order to provides a stimulating sensation to the user, to transmit current across different portions of the scalp, and/or for other benefit. In some embodiments, microcurrent module 932 applies current between the first electrode and second electrode of a same bristle (or more than one bristle). In some embodiments, the microcurrent module 932 applies current between a plurality of different bristles (i.e., one bristle is a positive electrode, and a second bristle is a negative electrode). In some embodiments, the microcurrent module 932 receives a signal from the contact sensing/moisture measurement module 934 (described below) that causes the microcurrent module 932 to start/stop application of an electrical current to the user's scalp.

Contact sensing/moisture measurement module 934, in some embodiments, measures an impedance of a user's scalp between a first electrode and a second electrode of a bristle (via return path to the handle), or between two bristles, e.g., when powered by the device 900. In embodiments in which power supply 904 is an alternating current power supply, contact sensing/moisture measurement module 934 measures impedance. In embodiments in which power supply 904 is a direct current power supply, contact sensing/moisture measurement module 934 measures resistance. Accordingly, the term “impedance” is used herein to mean both impedance (in AC embodiments) and resistance (in DC embodiments).

Measuring impedance of a user's scalp is useful because it 1) correlates to a moisture level and 2) indicates contact between one or more bristles with the scalp. Accordingly, in some embodiments, the contact sensing/moisture measurement module 934 provides a diagnostic of the moisture content and/or health of the user's scalp based upon the measured impedance. This diagnostic provides useful information to the user, e.g., signaling to the user whether further treatment is advisable. In some embodiments, the contact sensing/moisture measurement module 934 transmits a signal to one or more other modules verifying contact between at least one bristle and the scalp, e.g., the formulation module 930, microcurrent module 932, energy therapy module 936, haptic module 938, and/or vacuum module 940 in order to enable additional functionality.

In some embodiments, contact sensing/moisture measurement module 934 is communicatively coupled with one or more contact sensors 912 in order determine scalp contact. For example, in some embodiments, contact sensing/moisture measurement module 934 receives a signal from a dielectric contact sensor or from a piezoelectric sensor disposed on an end tip portion of the bristle, and based on that signal, determines whether there is scalp contact with the bristle (or a degree of scalp contact).

Energy therapy module 936 operates the electromagnetic energy sources 912 disposed on one or more bristles of the brush portion 908, in order to provide a soothing thermal treatment, to cure formulation applied to the user's scalp and/or hair, or for other advantage. Energy therapy module 936 is configured to modulate an intensity level, an “on/off” status, an “on” duration, and/or other features of one or more electromagnetic energy sources 912. Additionally, some embodiments are configured to emit different patterns of energy, i.e., the energy sources on different bristles are activated. In some embodiments, the activation/deactivation of one or more electromagnetic energy sources 912 is based upon a signal received from the contact sensing/moisture measurement module 934 indicative of contact between at least one bristle and the user's scalp. In other words, the electromagnetic energy source 912 are not activated unless the signal indicates contact with the scalp.

Haptic module 938 activates/deactivates one or more features of haptic system 918. For example, in some embodiments, the haptic module 938 activates one or more actuators 914 of the brush portion 908 to cause the bristles to oscillate back-and-forth along an x-, y-, and/or z-axis. In some embodiments, such movement is configured to massage the user's scalp, and/or to signal a device parameter (e.g., successful treatment cycle, end of treatment cycle, positive scalp contact, low formulation, low battery, etc.). In some embodiments, haptic module 938 activates/deactivates the haptic system 918 based upon a signal from contact sensing/moisture measurement module 934 (e.g., indicative of positive scalp contact).

Vacuum module 940 activates the vacuum system 920 in order to remove debris, excess formulation, and other matter from the user's scalp. In some embodiments, vacuum module 940 activates/deactivates the vacuum system 920 based upon a signal from contact sensing/moisture measurement module 934 (e.g., indicative of positive scalp contact).

An optional multiplexer 942 is operatively connected to the controller 906 and to the brush portion 908 in order to toggle different features of the device 900. As one example, each module represents an input, the brush portion 908 represents the output, and the multiplexer toggles between functions of the any of the module described above. In this way, the controller 906 utilizes the same electrodes 910 to perform a plurality of functions, e.g., current application, impedance measurement, and/or formulation application. In another embodiment, the multiplexer 942 functions as a demultiplexer, with numerous outputs thereof corresponding to different bristles of the brush portion 908. In this way, controller 906 is configured to execute one or more of: applying current to different bristles at different times for an improved stimulating effect; measuring impedance between different bristles and/or changing the bristles are powered in order to measure impedance (between electrodes of the same bristle) in order to determine scalp contact across a larger area of the brush portion 908 or to determine an average impedance value across a scalp area; to actuate actuators in different bristles for an improved massaging effect; and/or to deliver different functions to different bristles. For example, in some embodiments, multiplexer 942 enables controller 906 to apply current using bristles A+B, while measuring impedance using bristles C+D, and while simultaneously applying formulation and vacuuming with bristles A, B, C, and D.

For clarity, any two or more of the foregoing modules are configured to operate simultaneously, for improved user experience. For example, in some embodiments, the vacuum module 940 activates the vacuum system 920 simultaneously with the activation of the formulation dispenser 916 by the formulation module 930.

The foregoing configuration is representative, not limiting. Some embodiments include fewer or additional modules. Restated, all of the modules described herein are optional, such that embodiments of the devices described herein can have any combination of the foregoing modules. Further, in some embodiments, any of the foregoing modules are configured to communicate with one or more of the other modules.

Thus, the foregoing disclosure provides devices and bristles configured to provide a number of nonobvious advantages, whether alone or in combination, including: nebulizing a formulation from the bristles, applying current to a user's scalp, measuring impedance of a user's scalp, applying electromagnetic energy to a user's scalp, providing haptic stimulation to a user's scalp; and vacuuming debris and excess formulation.

The present application may also reference quantities and numbers. Unless specifically stated, such quantities and numbers are not to be considered restrictive, but representative of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The detailed description set forth above in connection with the appended drawings, where like numerals reference like elements, are intended as a description of various embodiments of the present disclosure and are not intended to represent the only embodiments. Each embodiment described in this disclosure is provided as a representative example or illustration and should not be construed as preferred or advantageous over other embodiments. The illustrative examples provided herein are not intended to be exhaustive or to limit the disclosure to the precise forms disclosed. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

Similarly, any steps described herein may be interchangeable with other steps, or combinations of steps, in order to achieve the same or substantially similar result. Generally, the embodiments disclosed herein are non-limiting, and the inventor contemplates that other embodiments within the scope of this disclosure may include structures and functionalities from more than one specific embodiment shown in the figures and described in the specification. In particular, features of any one embodiment may be adapted to any other embodiment except where expressly stated otherwise. Additionally, features prefaced with “in some embodiments,” are intended to communicate features that may be embodied in any other embodiment of the present disclosure. Furthermore, every feature described herein shall be understood to be applicable to all other contemplated embodiments except where expressly stated otherwise.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps or structural features have not been described in detail in order not to unnecessarily obscure various aspects of the present disclosure. Further, it will be appreciated that additional embodiments of the present disclosure may employ any combination of features described herein.

What is claimed is:

1. A brush portion configured for use with a handheld device, comprising:
a bristle disposed on a bristle base, the bristle comprising:
an elongate first electrode;
an elongate second electrode abutting the first electrode; and
an insulator disposed between the first electrode and the second electrode,
wherein the first electrode and the second electrode are electrically connectable to a first voltage source and a second voltage source of the device, respectively.

2. The brush portion of claim 1, further comprising a first plurality of fluid conduits extending through the first electrode and comprising a plurality of openings formed through at least one of an outer circumferential surface of the first electrode or an end tip surface of the first electrode.

3. The brush portion of claim 2, further comprising a second plurality of fluid conduits extending through the second electrode and comprising a second plurality of openings formed through at least one of an outer circumferential surface of the second electrode or an end tip surface of the second electrode.

4. The brush portion of claim 1, further comprising an electromagnetic energy source disposed on at least one of the first electrode or the second electrode, the electromagnetic energy source being configured to be electrically powered by the handheld device.

5. The brush portion of claim 4, wherein the electromagnetic energy source is configured to be electrically powered by the handheld device via the first electrode and the second electrode.

6. The brush portion of claim 1, further comprising at least one actuator disposed along the bristle in order to move the bristle when electrically powered by the handheld device.

7. The brush portion of claim 6, wherein the at least one actuator is at least partially formed of a piezoelectric material or a shape memory material.

8. The brush portion of claim 6, wherein the at least one actuator comprises a first actuator and a second actuator disposed at diametrically opposed locations of the bristle.

9. The brush portion of claim 1, wherein the first electrode is formed as a first half cylinder, and the second electrode is formed as a second half cylinder abutting the first half cylinder.

10. The brush portion of claim 9, further comprising a plurality of fluid conduits extending through at least one of the first electrode or the second electrode and comprising a plurality of openings formed through at least one of an outer circumferential surface of the bristle or an end tip surface of the bristle.

11. The brush portion of claim 10, further comprising at least one actuator disposed along the bristle in order to move the bristle when electrically powered by the handheld device.

12. The brush portion of claim 1, wherein the first electrode is formed as a first annular cylinder, and the second electrode is formed as a second cylinder disposed in an annular space of the first electrode.

13. The brush portion of claim 1, further comprising a plurality of fluid conduits extending through at least one of the first electrode or the second electrode and comprising a plurality of openings formed through at least one of an outer circumferential surface of the bristle or an end tip surface of the bristle.

14. The brush portion of claim 13, further comprising at least one actuator disposed along a length of the bristle, the at least one actuator being configured to move the bristle when electrically powered by the handheld device.

15. The brush portion of claim 1, wherein the first electrode is formed as a first annular half cylinder, the second electrode is formed as a second annular half cylinder abutting the first electrode, the bristle further comprising an inner cylinder disposed within an annular space formed by the first electrode and the second electrode.

16. A device, comprising:
a handle configured to receive a cartridge containing a formulation; and
the brush portion of claim 1 disposed on the handle.

17. The device of claim 16, wherein the handle comprises a controller electrically connected to the brush portion, wherein the controller includes logic that when executed by the controller causes the device to perform operations, including:
supplying the formulation from the cartridge to the bristle; and at least one of:
transmitting an electrical current between the first electrode and the second electrode; or
measuring an electrical impedance between the first electrode and the second electrode.

18. The device of claim 17,
further comprising a plurality of fluid conduits extending through at least one of the first electrode or the second electrode of the bristle,
wherein the controller includes logic that when executed by the controller causes the device to supply the formulation from the cartridge through the plurality of fluid conduits.

19. The device of claim 18,
further comprising at least one actuator disposed along the bristle,
wherein the controller includes logic that when executed by the controller causes the device to power the at least one actuator in order to move the bristle.

20. The device of claim 18,
further comprising an electromagnetic energy source disposed on an end tip portion of the bristle,
wherein the controller includes logic that when executed by the controller causes the device to power the electromagnetic energy source.
